(19) United States
(12) Patent Application Publication
(10) Pub. No.: US 2021/0215634 A1
(43) Pub. Date: Jul. 15, 2021

(54) COMPONENT SET FOR THE PRODUCTION OF READY-FOR-USE CHLORINE DIOXIDE SENSOR

(71) Applicant: ProMinent GmbH, Heidelberg (DE)

(72) Inventor: Klaus Koppert, Mannheim (DE)

(21) Appl. No.: 17/141,591

(22) Filed: Jan. 5, 2021

(30) Foreign Application Priority Data
Jan. 14, 2020 (DE) 10 2020 100 654.3

(51) Int. Cl. G01N 27/413

(52) U.S. Cl. CPC G01N 27/413

(57) ABSTRACT

To provide a component set for the production of a ready-for-use chlorine dioxide sensor which affords a chlorine dioxide sensor containing a strongly acid electrolyte which has exactly the low pH-value in the range of 1.1 to 1.5, that is desired for use in the chlorine dioxide sensor, according to the invention there is proposed a component set characterised in that it includes an amperometric measuring cell having an electrolyte chamber and a working electrode arranged therein and a reference electrode arranged therein, and at least one container containing a liquid standard electrolyte and at least one container containing a solid acid additive.

The present invention concerns a component set for the production of a ready-for-use chlorine dioxide sensor. The invention further concerns a process for the production of a chlorine dioxide sensor using the component set according to the invention.

Chlorine dioxide (ClO₂) is an effective disinfectant for water disinfection. For those purposes chlorine dioxide is used inter alia in the area of communal drinking and sewerage treatment, in industrial operations, by drinks manufacturers and in catering and hospitals to achieve reliable disinfection and sterilisation of drinking, service and process water. In that situation the chlorine dioxide concentration always has to be reliably set to achieve the desired sterilisation effect. For that purpose use is made of chlorine dioxide sensors which typically have an amperometric measuring cell with an electrolyte chamber in which a working electrode and a reference electrode are arranged.

A strongly acid electrolyte with a pH-value of markedly below 2 is used as the electrolyte which surrounds the reference electrode in the electrolyte chamber. That is required to achieve a selectivity of <5% in relation to chlorine which is present in the water at the same time. As in the reduction reaction of the chlorine dioxide at the working electrode surface protons (H⁺) are consumed in accordance with the following reaction:

4H⁺+ClO₂+5e⁻═Cl⁻+2H₂O

the electrolyte is set to be somewhat more acid than would be necessary in order in that way to achieve a long lifetime and to counteract the slow drift of the pH-value in the direction of pH 2. Typically the strongly acid electrolyte is set to a pH-value in the range of 1.1 to 1.5 and that is usually effected by suitably dosed addition of hydrochloric acid (HCl).

The pH-value of 2 represents a limit, above which the above-indicated reaction no longer takes place stoichiometrically. A correlation between sensor current and chlorine dioxide concentration in the water is then no longer a given. To achieve a long lifetime for the chlorine dioxide sensor it is therefore entirely crucial that the pH-value of the electrolyte initially used is as low as possible.

Conventional chlorine dioxide sensors are dispatched in a component set which comprises the amperometric measuring cell and a container in which the strongly acid electrolyte is contained. To make the chlorine dioxide sensor ready for use the user on site only still has to introduce the strongly acid electrolyte into the electrolyte chamber of the amperometric measuring cell and to calibrate the chlorine dioxide sensor after the expiry of a certain start-up phase.

In the case of conventional strongly acid electrolytes which are dispatched to the user the pH-value is therefore set by the manufacturer and, depending on the respective duration of storage, transport and stocking on the part of the user several weeks and months can pass under some circumstances, within which the pH-value of the strongly acid electrolyte solution can increase significantly, for example by reaction with the carbon dioxide contained in the air. That effect can be observed in particular in the situations in which the strongly acid electrolyte is subjected to severe shaking, fluctuating temperature conditions and/or widely varying pressure conditions during transport and/or storage.

There is therefore a need for a component set for the production of a ready-for-use chlorine dioxide sensor with which a chlorine dioxide sensor is obtained, which contains a strongly acid electrolyte, which has exactly the low pH-value in the range of 1.1 to 1.5 that is desired for use in the chlorine dioxide sensor.

According to the invention that object is attained by a component set for the production of a ready-for-use chlorine dioxide sensor which is characterised in that said component set includes the following components:

a) an amperometric measuring cell having an electrolyte chamber and a working electrode arranged in the electrolyte chamber and a reference electrode arranged in the electrolyte chamber,

b) at least one container containing a liquid standard electrolyte, and

c) at least one container which contains a solid acid additive.

By virtue of the provision according to the invention of a component set which besides a liquid standard electrolyte also contains a solid acid additive a user who has obtained the component set can produce therefrom, with few manipulations, a strongly acid electrolyte which has the low pH-value in the range of 1.1 to 1.5, that is desired for use in the chlorine dioxide sensor. For that purpose the user only has to add the solid acid additive provided according to the invention to the liquid standard electrolyte provided according to the invention and dissolve same therein.

Providing the component set according to the invention entails the advantage that the strongly acid electrolyte solution can be produced by the user on site, thereby ensuring that when using the appropriate amount of acid additive the solution has the desired low pH-value. The advantage is in particular that the desired pH-value occurs in situ, that is to say immediately prior to calibration and first measurement. With the conventional strongly acid electrolyte dispatched to the user the pH-value was set by the manufacturer and depending on the respective duration involved in storage, transport and stocking on the part of the user, several weeks and months can elapse under some circumstances, within which the pH-value of the strongly acid electrolyte solution can increase significantly, as was already set forth hereinbefore. In the case of the present invention that danger does not occur by virtue of production in situ, and by virtue of the provision of the acid additive in the form of a solid the reaction with the carbon dioxide contained in the air, that is possible with acid solutions, also does not occur here.

Besides the above-indicated advantage the component set according to the invention affords the additional advantage that it can be dispatched by the manufacturer to the user without incurring the tariffs incurred for the transport of hazardous substances. In many countries liquids with a pH-value of <2 are considered as a hazardous substance for which particularly strict transport regulations apply. That has in particular consequences in terms of the transport costs as the transport of hazardous substances is markedly more expensive than the transport of substances which are not classified as hazardous substances. That applies in particular to transport with an aircraft.

With the technical solution proposed according to the invention the transport costs for chlorine dioxide sensors can be markedly reduced, the reason for this being that in the component set according to the invention a liquid standard electrolyte is provided, the pH-value of which does not fall in the range which would lead to it being classified as a hazardous substance. That also applies to the solid acid additive which is additionally contained in the component set according to the invention and with which the user, after receiving the component set, can produce with few manipulations by mixing with the liquid standard electrolyte a strongly acid electrolyte which has the low pH-value in the range of 1.1 to 1.5, that is desired for use in the chlorine dioxide sensor.

The term “component set” is used in accordance with the invention to denote that this involves a predetermined combination of individual components representing separate individual parts which only when brought together give the actual product. In the present case these components are the amperometric measuring cell, the liquid standard electrolyte contained in a container and the solid acid additive contained in a further container, the combination thereof as its product giving the ready-for-use chlorine dioxide sensor.

The measuring cell included in the component set according to the invention has an electrolyte chamber which is filled with electrolyte in the ready-for-use state of the chlorine dioxide sensor. Arranged in the electrolyte chamber are a working electrode and a reference electrode which are both immersed in the electrolyte in the ready-for-use state of the chlorine dioxide sensor. The measuring cell encloses the electrolyte in fluid-tight relationship on all sides, in which respect a valve opening can be provided for pressure equalisation. At one side the measuring cell has a diaphragm, by way of which the analyte can diffuse into the electrolyte chamber and come into contact with the working electrode so that the measuring cell produces an electrical signal, the signal strength of which correlates with the level of a chlorine dioxide concentration measured by the sensor. In the case of the amperometric measuring cell involved here a reduction current is measured at the working electrode while an electrochemical potential which is constant in respect of time is applied. The measured reduction current is directly proportional to the concentration of the chlorine dioxide which is reacted in accordance with the foregoing equation at a constant temperature.

The liquid standard electrolyte and the solid acid additive of the component set according to the invention are respectively provided in separate containers. The containers used according to the invention have an opening closable with a cover, wherein in the case of the component set according to the invention the containers containing the liquid standard electrolyte and the solid acid additive respectively are closed for the purposes of storage or transport.

Preferably the containers used according to the invention comprise a plastic. Certain embodiments of the containers comprise polyethylene (PE). In certain embodiments the container cover is a screw cover which can be fluid-tightly screwed on to the opening of the container.

The volume of the container in which the standard electrolyte is contained is in the range of 10 to 250 ml. In particular embodiments the volume is in the range of 10 to 100 ml or 10 to 50 ml or in the range of 50 to 100 ml.

The volume of the container in which the solid acid additive is contained is in the range of 2 to 15 ml. In certain embodiments the volume of that container is 2 to 10 ml or 2 to 5 ml. The term “liquid standard electrolyte” is used in accordance with the present invention to denote that this involves an aqueous solution of an electrolyte which is suitable for use in the electrolyte chamber of an amperometric measuring cell. In a particular embodiment the liquid standard electrolyte is an aqueous solution of an alkali metal chloride. Preferably the alkali metal chloride contained in the standard electrolyte is selected from lithium chloride (LiCl), sodium chloride (NaCl) or potassium chloride (KCl).

In certain embodiments that alkali metal chloride solution is in a concentration in the range of 0.2 to 3 mol/l. In certain embodiments of the invention the standard electrolyte is an aqueous solution of alkali metal chloride which preferably involves a concentration in the range of 0.2 to 0.5 mol/l.

The term “liquid” is to be interpreted here as meaning that the material identified thereby is in the liquid aggregate state at ambient temperature (20° C.). In contrast thereto the term “solid” is to be interpreted as meaning that material identified thereby is in the solid aggregate state at ambient temperature (20° C.).

The term “solid acid additive” in accordance with the foregoing definition is to be interpreted as being a chemical substance which is in the form of a solid at ambient temperature and which upon solution in water or an aqueous solution leads to a reduction in the pH-value. In certain embodiments of the invention the solid acid additive is an alkali metal hydrogen sulphate. In certain embodiments lithium hydrogen sulphate (LiHSO₄), sodium hydrogen sulphate (NaHSO₄) or potassium hydrogen sulphate (KHSO₄) is used as the solid acid additive.

In order to make the production of the strongly acid electrolyte with the desired pH-value as easy as possible for the user and to exclude possible calculation and/or measurement errors in certain embodiments of the invention the stoichiometric amount of standard electrolyte in the at least one container which contains a standard electrolyte and the stoichiometric amount of acid additive in the at least one container which contains an acid additive are so matched to each other that either:

a) a mixture of the total stoichiometric amount of the acid additive contained in the container with the total stoichiometric amount of the standard electrolyte contained in the container gives a strongly acid electrolyte with a pH-value in the range of 1:1 to 1:5, or

b) a mixture of a whole-numbered volume or mass proportion of the acid additive contained in the container with the total stoichiometric amount of the standard electrode contained in the container gives a strongly acid electrolyte with a pH-value in the range of 1.1 to 1.5, or

c) a mixture of the total stoichiometric amount of the acid additive contained in the container with a whole-numbered volume or mass proportion of the standard electrolyte contained in the container gives a strongly acid electrolyte with a pH-value in the range of 1.1 to 1.5.

With the component set provided according to the invention a strongly acid electrolyte with a pH-value in the range of 1.1 to 1.5 can be quickly and easily obtained by the user without any calculations and technically labourious measurements. In certain embodiments in that way with the component set according to the invention a strongly acid electrolyte with a pH-value in the range of 1.2 to 1.4 is produced. In further particular embodiments of the invention the pH-value obtained with the component set for the strongly acid electrolyte is at pH of 1.3+/−0.15.

In certain embodiments of the invention the component set includes two, three, four or five containers which contain the acid additive in an amount with which the total stoichiometric amount of the standard electrolyte contained in the at least one standard electrolyte container or a whole-numbered volume or mass proportion of the standard electrolyte contained in the at least one standard electrolyte container can be converted to a strongly acid electrolyte with a pH-value in the above-mentioned ranges.

In certain embodiments of the invention the component set provided additionally includes a dosage instruction which either:

a) contains the instruction that the total volume or the total mass of acid additive in the at least one container containing said acid additive must be mixed with the total volume or the total mass of the standard electrolyte in the at least one container containing said standard electrolyte to obtain a strongly acid electrolyte with a pH-value in the range of 1.1 to 1.5, or

b) contains the instruction that a whole-numbered volume or mass proportion of the acid additive from the at least one container containing said acid additive must be mixed with the total volume or the total mass of the standard electrolyte in the at least one container containing said standard electrolyte in order to obtain a strongly acid electrolyte with a pH-value in the range of 1.1 to 1.5, or

c) contains the instruction that the total volume or the total mass of the acid additive from the at least one container containing said acid additive must be mixed with a whole-numbered volume or mass proportion of the standard electrolyte in the at least one container containing said standard electrolyte in order to obtain a strongly acid electrolyte with a pH-value in the range of 1.1 to 1.5.

According to the invention the dosage instruction can be provided on a sheet of paper or the like. Examples of this are a leaflet and/or an openable label placed on one of the containers.

The description of the instruction can be provided in text form and/or in the form of a pictogram. In certain embodiments of the invention to provide support the dosage instruction contains numerical, letter, symbol and/or colour codes which coincide with corresponding number, letter, symbol and/or colour codes on the containers of the component set.

In certain embodiments of the invention the dosage instruction includes in text and/or pictogram form the instruction that the acid additive is to be shaken into the container in which the standard electrolyte is contained and that then that container is to be shaken for such a period of time until the solid acid additive has dissolved.

In certain embodiments of the invention the component set includes additional constituents which can be used for the production of a ready-for-use chlorine dioxide sensor, like for example a cable for connection of the chlorine dioxide sensor to the corresponding fitting.

In an alternative embodiment the component set according to the invention comprises a transport packaging which is closable on all sides and in which the amperometric measuring cell, the container which contains a standard electrolyte and the container in which the solid acid additive is contained are arranged. Optionally the transport packaging additionally includes the above-described dosage instruction, further containers in which a solid acid additive is contained and/or a cable for connecting the chlorine dioxide sensor to a measuring fitting.

In certain embodiments the components are so arranged in the transport packaging that they are fixed with a slight play in the closed transport packaging. In a particular embodiment at least one suitably shaped polystyrene body is provided for the purposes of fixing in the transport packaging.

In alternative embodiments of the invention the transport packaging can in addition also contain further components which can be useful in connection with the set up and use of a chlorine dioxide sensor.

According to the invention there is also claimed a refill set for a chlorine dioxide sensor which is characterised in that it includes at least one container which contains a liquid standard electrolyte and at least one container which contains a solid acid additive, wherein the stoichiometric amount of standard electrolyte in the at least one container which contains a standard electrolyte and the stoichiometric amount of acid additive in the at least one container which contains an acid additive are so matched to each other that either:

a) a mixture of the total stoichiometric amount of the acid additive contained in the container with the total stoichiometric amount of the standard electrolyte contained in the container gives a strongly acid electrolyte with a pH-value in the range of 1.1 to 1.5, or

b) a mixture of a whole-numbered volume or mass proportion of the acid additive contained in the container with the total stoichiometric amount of the standard electrode contained in the container gives a strongly acid electrolyte with a pH-value in the range of 1.1 to 1.5, or

c) a mixture of the total stoichiometric amount of the acid additive contained in the container with a whole-numbered volume or mass proportion of the standard electrolyte contained in the container gives a strongly acid electrolyte with a pH-value in the range of 1.1 to 1.5.

According to the invention there is also claimed a process for the production of a chlorine dioxide sensor which is characterised in that the chlorine dioxide sensor is created from the component set of the above-described kind according to the invention. In a given embodiment of the process according to the invention for production of the chlorine dioxide sensor a part of the standard electrolyte contained in the at least one container is mixed with at least a part of the acid additive contained in the at least one container to produce a strongly acid electrolyte with a pH-value in the range of 1.1 to 1.5 or with a pH-value in the one of the above-specified narrower ranges.

In a particular embodiment of the invention the chlorine dioxide sensor is produced by operating in accordance with one of the above-described instructions to produce the strongly acid electrolyte with a pH-value in the desired range.

In the implementation of the process claimed in accordance with the invention preferably a whole-numbered quantitative proportion of the strongly acid electrolyte produced or indeed the total amount of the strongly acid electrolyte produced is introduced into the electrolyte chamber of the amperometric measuring cell.

To provide for use of a strongly acid electrolyte solution which is as fresh as possible in certain embodiments of the process according to the invention the chlorine dioxide sensor is calibrated within at most 24 hours after the strongly acid electrolyte was produced and introduced into the electrolyte chamber of the amperometric measuring cell.

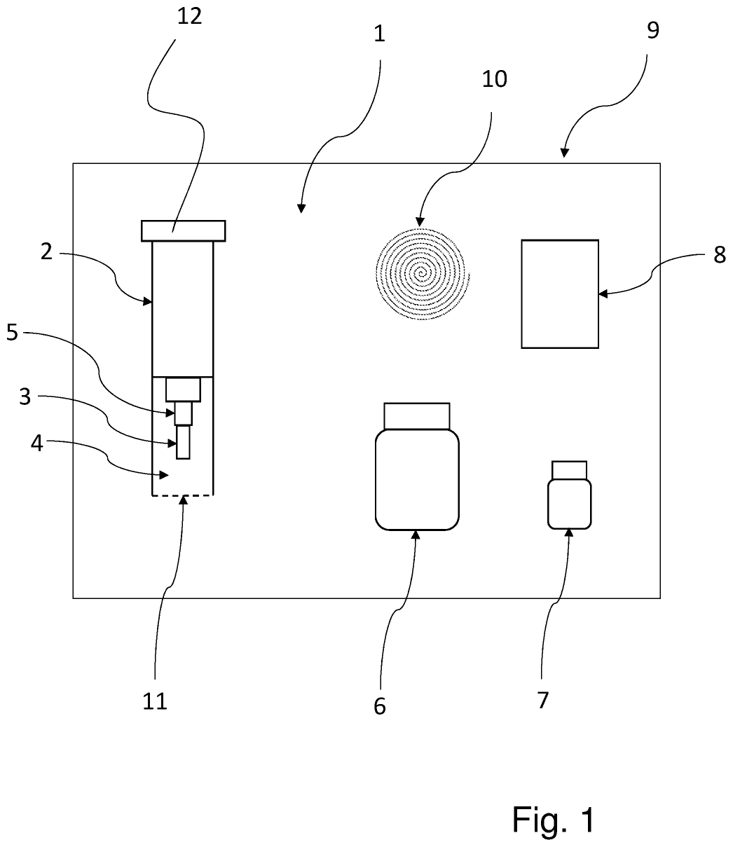

FIG. 1 shows a given embodiment of the component set according to the invention with transport packaging.

FIG. 1 shows a specific embodiment of the present invention in which the component set 1 according to the invention is arranged in a transport packaging 9. In the embodiment illustrated here the component set according to the invention includes an amperometric measuring cell 2, a container 6 for the standard electrolyte, a container 7 for the acid additive, a dosage instruction 8 and a cable 10 for connection of the ready-for-use chlorine dioxide sensor by way of the connection 13 to a measuring fitting (not shown). In the embodiment shown here the component set 1 is arranged in a transport packaging 9 which is closed on all sides.

The amperometric measuring cell 2 illustrated here has an electrolyte chamber 4 and a working electrode 3 arranged therein and a reference electrode 5 arranged therein. Disposed at the lower end is a diaphragm 11, by way of which analyte can penetrate into the electrolyte chamber of the amperometric measuring cell to come into contact there with the working electrode 3. Disposed at the upper end is the connection 12, by way of which the ready-for-use chlorine dioxide sensor can be connected to a measuring fitting (not shown) with the cable 10.

The containers 6 and 7 illustrated here are screw cover vessels of polyethylene (PE).

The dosage instruction 8 illustrated here is a leaflet of paper, containing in text form the instruction in accordance with which the strongly acid electrolyte with a pH-value in the range of 1.3+/−0.15 can be produced from the standard electrolyte contained in the container 6 and the solid acid additive contained in the container 7.

1. A component set (1) for the production of a ready-for-use chlorine dioxide sensor, characterised in that the component set includes the following components:
a) an amperometric measuring cell (2) having an electrolyte chamber (4) and a working electrode (3) arranged in the electrolyte chamber (4) and a reference electrode (5) arranged in the electrolyte chamber (4),
b) at least one container (6) containing a liquid standard electrolyte, and
c) at least one container (7) which contains a solid acid additive.

2. A component set according to claim 1 characterised in that the stoichiometric amount of standard electrolyte in the at least one container which contains a standard electrolyte and the stoichiometric amount of acid additive in the at least one container which contains an acid additive are so matched to each other that either:
a) a mixture of the total stoichiometric amount of the acid additive contained in the container with the total stoichiometric amount of the standard electrolyte contained in the container gives a strongly acid electrolyte with a pH-value in the range of 1.1 to 1.5, or
b) a mixture of a whole-numbered volume or mass proportion of the acid additive contained in the container with the total stoichiometric amount of the standard electrode contained in the container gives a strongly acid electrolyte with a pH-value in the range of 1.1 to 1.5, or
c) a mixture of the total stoichiometric amount of the acid additive contained in the container with a whole-numbered volume or mass proportion of the standard electrolyte contained in the container gives a strongly acid electrolyte with a pH-value in the range of 1.1 to 1.5.

3. A component set according to claim 1 characterised in that the component set includes 2, 3, 4 or 5 containers which contain the acid additive in an amount with which the total stoichiometric amount of the standard electrolyte contained in the at least one standard electrolyte container or a whole-numbered volume or mass proportion of the standard electrolyte contained in the at least one standard electrolyte container can be converted to a strongly acid electrolyte with a pH-value in the range of 1.1 to 1.5.

4. A component set according to claim 1 characterised in that the standard electrolyte is an aqueous solution of alkali metal chloride.

5. A component set according to claim 1 characterised in that the acid additive is an alkali metal hydrogen sulphate.

6. A component set according to claim 1 characterised in that it additionally includes a dosage instruction (8) which either
a) contains the instruction that the total volume or the total mass of acid additive in the at least one container containing said acid additive must be mixed with the total volume or the total mass of the standard electrolyte in the at least one container containing said standard electrolyte to obtain a strongly acid electrolyte with a pH-value in the range of 1.1 to 1.5, or
b) contains the instruction that a whole-numbered volume or mass proportion of the acid additive from the at least one container containing said acid additive must be mixed with the total volume or the total mass of the standard electrolyte in the at least one container containing said standard electrolyte in order to obtain a strongly acid electrolyte with a pH-value in the range of 1.1 to 1.5, or
c) contains the instruction that the total volume or the total mass of the acid additive from the at least one container containing said acid additive must be mixed with a whole-numbered volume or mass proportion of the standard electrolyte in the at least one container containing said standard electrolyte in order to obtain a strongly acid electrolyte with a pH-value in the range of 1.1 to 1.5.

7. A component set according to claim 1 characterised in that the component set is arranged in a transport packaging (9) which is closable on all sides.

8. A refill set for a component set according to claim 1 characterised in that the refill set includes at least one container which contains a liquid standard electrolyte and at least one container which contains a solid acid additive, wherein the stoichiometric amount of standard electrolyte in the at least one container which contains a standard electrolyte and the stoichiometric amount of acid additive in the at least one container which contains an acid additive are so matched to each other that either:
a) a mixture of the total stoichiometric amount of the acid additive contained in the container with the total stoichiometric amount of the standard electrolyte contained in the container gives a strongly acid electrolyte with a pH-value in the range of 1:1 to 1:5, or
b) a mixture of a whole-numbered volume or mass proportion of the acid additive contained in the container with the total stoichiometric amount of the standard electrode contained in the container gives a strongly acid electrolyte with a pH-value in the range of 1.1 to 1.5, or
c) a mixture of the total stoichiometric amount of the acid additive contained in the container with a whole-numbered volume or mass proportion of the standard electrolyte contained in the container gives a strongly acid electrolyte with a pH-value in the range of 1.1 to 1.5.

9. A process for the production of a chlorine dioxide sensor characterised in that the chlorine dioxide sensor is created from the component set according to claim 1.

10. A process according to claim 9 wherein at least a part of the standard electrolyte contained in the at least one container is mixed with at least a part of the acid additive contained in the at least one container to produce a strongly acid electrolyte with a pH-value in the range of 1.1 to 1.5.

11. A process according to claim 9 characterised in that in accordance with a dosage instruction (8) which either
a) contains the instruction that the total volume or the total mass of acid additive in the at least one container containing said acid additive must be mixed with the total volume or the total mass of the standard electrolyte in the at least one container containing said standard electrolyte to obtain a strongly acid electrolyte with a pH-value in the range of 1.1 to 1.5, or
b) contains the instruction that a whole-numbered volume or mass proportion of the acid additive from the at least one container containing said acid additive must be mixed with the total volume or the total mass of the standard electrolyte in the at least one container containing said standard electrolyte in order to obtain a strongly acid electrolyte with a pH-value in the range of 1.1 to 1.5, or
c) contains the instruction that the total volume or the total mass of the acid additive from the at least one container containing said acid additive must be mixed with a whole-numbered volume or mass proportion of the standard electrolyte in the at least one container containing said standard electrolyte in order to obtain a strongly acid electrolyte with a pH-value in the range of 1.1 to 1.5;
a strongly acid electrolyte with a pH-value in the range of 1.1 to 1.5 is produced.

12. A process according to claim 9 characterised in that a whole-numbered quantitative proportion of the strongly acid electrolyte produced or the total amount of the strongly acid electrolyte produced is introduced into the electrolyte chamber of the amperometric measuring cell.

13. A process according to claim 12 characterised in that the chlorine dioxide sensor is calibrated within at most 24 hours after the strongly acid electrolyte was produced and introduced into the electrolyte chamber of the amperometric measuring cell.
